(19) United States
(12) Patent Application Publication
(10) Pub. No.: US 2021/0262964 A1
(43) Pub. Date: Aug. 26, 2021

(54) NANOTUBE SENSORS AND RELATED METHODS

(71) Applicant: EXHALENT DIAGNOSTICS, INC., Taylorsville, UT (US)

(72) Inventors: Blain Chappell, Taylorsville, UT (US); Keith Prisbrey, Salt Lake City, UT (US)

(21) Appl. No.: 17/180,517

(22) Filed: Feb. 19, 2021

Related U.S. Application Data
(60) Provisional application No. 62/979,268, filed on Feb. 20, 2020.

(51) Int. Cl. G01N 27/12; G01N 33/543; G01N 1/44

(52) U.S. Cl. CPC G01N 27/127; B82Y 5/00; G01N 1/44; G01N 33/5438

(57) ABSTRACT

An example of a nanotube sensor includes an array of nanotubes formed on a substrate. The nanotube sensor includes a power source connected to and configured to provide electrical power (e.g., a voltage) to the array of nanotubes. The nanotube sensor also includes an electrical sensor configured to detect at least one electrical characteristic of the array of nanotubes when the electrical power is provided to the array of nanotubes. The nanotube sensor further includes electrical circuitry including at least one processor and memory. The electrical circuitry is connected to the electrical sensors and is configured to receive the detected electrical characteristic detected by the electrical sensor. The memory of the electrical circuitry includes one or more machine learning algorithms stored therein that, when executed by the processor, allows the electrical circuitry to analyze the detected electrical characteristic to determine if one or more markers are detected.

### CROSS-REFERENCE TO RELATED APPLICATION

This application is a nonprovisional patent application of and claims priority to U.S. Provisional Patent Application No. 62/979,268 filed Feb. 20, 2020, and entitled “Nanotube Sensors and Related Methods,” the disclosure of which is hereby incorporated by reference in its entirety.

### BACKGROUND

Identifying and developing noninvasive techniques for detection of pathogenic conditions of the human body is an area of growing interest, especially as it relates to rapid biosensing and the diagnosis of diseases at the point of care (POC). These noninvasive techniques are preferred for POC diagnosis as handling of traditional samples, such as blood, requires special skills and exposes the health care worker to possible blood borne pathogens. Ideally, non-invasive methods of diagnostics reduce this risk. Researchers have focused on screening external biological samples (i.e. saliva, urine, hair, sweat, and sputum) for markers (e.g., biomarkers) that indicate conditions such has diabetes, dehydration, and other diseases. Examples of typical markers include antigens, antibodies, or proteins that require a liquid environment for analysis. Consequently, diagnosis of diseases typically requires a liquid biological sample such as those mentioned above. However, one class of markers that is known to have associations with certain diseases, but has found limited use as a diagnostic tool, is volatile organic markers. Volatile organic markers have been associated with different chronic and infectious diseases including tuberculosis (TB).

As a specific example, conventional methods for tuberculosis (TB) detection are traditionally performed in laboratories or hospitals. For example, the most common method for diagnosis of TB is the acid fast staining of clinical material, which is then followed by a sputum smear microscopy test. However, a disadvantage with the sputum smear test is its poor sensitivity, which is estimated to be at 70%. Additionally, the sensitivity of sputum smear spectroscopy in settings in the field has been shown to be much lower (e.g. 35%), especially in populations that have high rates of TB and HIV co-infection. Furthermore, drug susceptibility analysis of the mycobacterium cannot be determined from microscopy testing. This assessment is useful in determining the appropriate course of treatment for the patient. Culturing techniques are typically used for this type of analysis.

Culturing of mycobacterium from sputum samples is a more sensitive technique. Sputum samples are collected and cultured in either solid media or liquid media looking for the presence of the mycobacterium. Drug resistant strains can be determined using this technique. However, this methodology takes time to conduct (3-4 weeks for solid cultures, and 10-14 days for liquid cultures), which makes it difficult to employ in low resource settings that are typically far from testing facilities. Recently, other technologies have been developed including fluorescence microscopy for smear tests (10% more sensitive than light microscopy), LED fluorescent microscopy for inexpensive imaging equipment that can be used in the field without the need for a darkroom, and rapid culturing techniques to reduce incubation time. Despite the improvements that have been made in TB diagnosis, no simple inexpensive POC test is currently available. Additionally, a need exists for general POC tests designed to detect any number of diseases, viruses, other health conditions, as well as substance abuse.

### SUMMARY

Nanotube sensors and related systems and methods for optimizing the use, functionality, and accuracy of the nanotube sensors are disclosed herein. An example of a nanotube sensor includes an array of nanotubes (e.g., a plurality of nanotubes) formed on a substrate. The nanotube sensors can include, for example, non-functionalized titanium dioxide (TiO₂) nanotubes grown on a titanium plate. The nanotube sensor includes a power source connected to and configured to provide electrical power (e.g., a voltage) to the array of nanotubes. The nanotube sensor also includes an electrical sensor configured to detect at least one electrical characteristic of the array of nanotubes when the electrical power is provided to the array of nanotubes. The nanotube sensor further includes electrical circuitry including at least one processor and memory (e.g., non-transitory memory). The electrical circuitry is connected to the electrical sensors and is configured to receive the detected electrical characteristic detected by the electrical sensor. The memory of the electrical circuitry includes one or more machine learning algorithms stored therein that, when executed by the processor, allows the electrical circuitry to analyze the detected electrical characteristic to determine if one or more markers (e.g., biomarker) are detected.

Features from any of the disclosed embodiments can be used in combination with one another, without limitation. In addition, other features and advantages of the present disclosure will become apparent through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a cross-sectional view of a nanotube that can be used in any of the arrays of nanotubes disclosed herein, according to one exemplary embodiment.

FIG. 1B is a schematic illustration of a nanotube sensor 200, according to one exemplary embodiment.

FIG. 2 is a graph illustrating the detected current of a sample of Working Example 3 that included methamphetamine

FIG. 3 is a graph illustrating the detected current of a sample of Working Example 3 that included methamphetamine and a sample of Working Example 3 that did not include methamphetamine.

### DETAILED DESCRIPTION

Nanotube sensors and related systems and methods for optimizing the use, functionality, and accuracy of the nanotube sensors are disclosed herein. An example of a nanotube sensor includes an array of nanotubes (e.g., a plurality of nanotubes) formed on a substrate. The nanotube sensors can include, for example, non-functionalized titanium dioxide (TiO₂) nanotubes grown on a titanium plate. The nanotube sensor includes a power source connected to and configured to provide electrical power (e.g., a voltage) to the array of nanotubes. The nanotube sensor also includes an electrical sensor configured to detect at least one electrical characteristic of the array of nanotubes when the electrical power is provided to the array of nanotubes. The nanotube sensor further includes electrical circuitry including at least one processor and memory (e.g., non-transitory memory). The electrical circuitry is connected to the electrical sensors and is configured to receive the detected electrical characteristic detected by the electrical sensor. The memory of the electrical circuitry includes one or more machine learning algorithms stored therein that, when executed by the processor, allows the electrical circuitry to analyze the detected electrical characteristic to determine if one or more markers are detected. The biomarkers can include any chemical compound, such as one or more biomarkers and/or one or more non-biomarkers. Examples of chemical compounds that can be detected include, but are not limited to, biomarkers for tuberculosis (e.g., methyl nicotinate and p-anisate), severe acute respiratory syndrome coronavirus 2 (“COVID 19”), Escherichia coli, listeria, methamphetamine, explosive compounds, biomarkers for colorectal cancer (e.g., cyclohexane, methylcyclohexane, 1,3-methylbenzene, and decanal), ammonia, nitrates, and tetrahydrocannabinol.

A method of using the nanotube sensor can include providing a sample to the nanotube sensor. The sample can include, but is in no way limited to, breath, air, a vaporized material, another gaseous sample, a liquid sample, or molecules dissolved or otherwise dispersed in a gaseous or liquid material. In particular, providing the sample includes exposing the array of nanotubes to the sample. The surfaces of the nanotubes can bind or otherwise react with one or more markers that are present in the sample which changes the electrical resistance of the array of nanotubes. The power source provides electrical power to the array of nanotubes which is then used to detect the change in the electrical resistance of the array of nanotubes. For example, the power source can provide electrical power at a plurality of different voltages, and the electrical current at each of the plurality of different voltages is detected. The electrical circuitry receives signals (e.g., electrical current) from the array of nanotubes and, using the machine learning algorithms, analyzes the signals to determine if one or more markers are present in the sample. The machine learning algorithm can allow the electrical circuitry to perform at least one of the following functions: detect the marker even if the electrical circuitry does not initially know which marker(s) can be present in the sample (e.g., the nanotube sensor can be used to simultaneously test for a plurality of markers), minimize or eliminate error caused by noise, or detect the marker even if volatile compounds in the sample interfere with the signal.

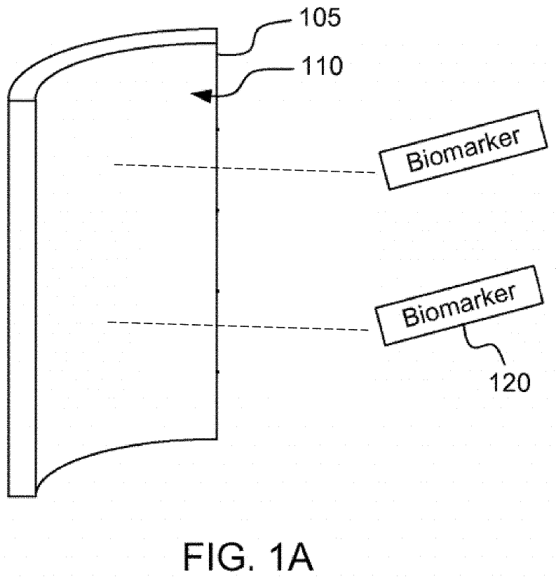

FIG. 1A is a cross-sectional view of a nanotube 105 that can be used in any of the arrays of nanotubes disclosed herein, according to an embodiment. The nanotube 105 can be formed on a substrate (not shown) and can have an interior surface 110 and/or exterior surface (not shown, obscured). In a preferred embodiment, the interior surface 110 and/or the exterior surface is not functionalized which can provide a number of advantages, including but in no way limited to, making the manufacturing of the nanotubes 105 simple and quicker, increasing the sensitivity of the nanotube sensor that includes the nanotubes, or allowing the nanotubes to bind with or otherwise react with a wider variety of markers 120 than if the nanotubes were functionalized with one or more metal ions. However, in an exemplary embodiment, the interior surface 110 and/or the exterior surface can be functionalized with at least one metal ion to improve the sensitivity of the nanotube sensor that includes the nanotubes 105 with regard to a selected marker. In such an embodiment, a metal ion is chosen for selective binding with a specific marker 120.

In general, the nanotube sensor including the nanotube 105 and methods for using the nanotube sensor include detecting a change in electric current across the nanotube 105. When the surface of the nanotube 105 binds or otherwise reacts with the marker 120 (shown with dashed lines), the electrical resistance of the nanotube 105 can change. When a voltage is applied to the nanotube 105, the change in resistance can be detected as a change in a detected electrical current, or vice versa.

FIG. 1B is a schematic illustration of a nanotube sensor 200, according to one example. The nanotube sensor 200 includes a housing 205 which can provide a supportive platform and a physical protection to at least some of the components of the nanotube sensor 200. As used herein, external components of the nanotube sensor 200 include components that are spaced from the housing 205 and internal components include components that are disposed in or on the housing 205. The housing 205 includes openings for an intake 210 and an outlet 215. The intake 210 directs the sample (e.g., gaseous sample) into an interior space 220 of the nanotube sensor 200, while the outlet 215 allows the sample and excess fluid to exit the housing 205. Various additional components can be oriented within the interior space 220 of the housing 205. For example, a filter 225 can be oriented to remove particulates from sample fluid after entry through the intake 210. An optional concentrator 230 can be used to concentrate gases and/or vapors, and to increase sampling signals. Further, an array 235 of nanotubes 240, each of which can be the same as nanotube 105 of FIG. 1A, can be oriented along a path of the sample which enters the housing 205. Although specific dimensions can vary considerably, the length of the housing 205 can often range from about 8 cm to about 10 cm. The array 235 of nanotubes 240 can be connected to a power source 245 and an electrical sensor 250 (e.g., current sensor, voltage sensor, multimeter, potentiostat, etc.). The power source 245 and the electrical sensor 250 can be internal components of the nanotube sensor 200, in which case the power source 245 and the electrical sensor 250 are, according to one example, located within the housing 205. Alternatively, one or both of the power source 245 and the electrical sensor 250 can be external and can connect to the rest of the nanotube sensor 200 through any suitable connection including wired or wireless power and communication.

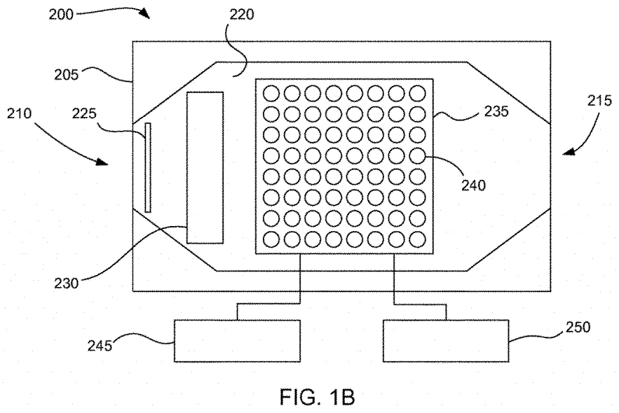

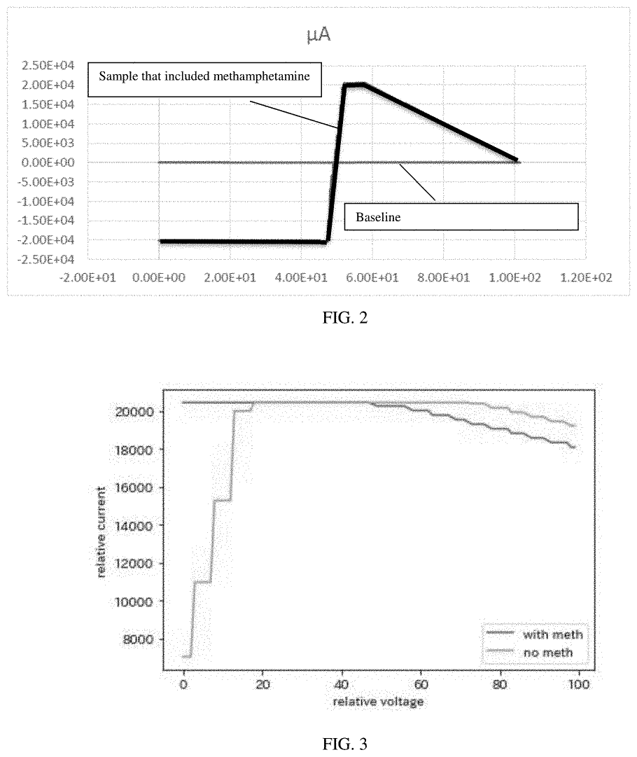

Electrical power (e.g., bias voltage) is applied across the array 235 using the power source 245. For example, a set of electrodes can be oriented to contact the array 235 at remote locations from one another. Such electrodes can then be wired to the power source 245. The electrodes, in some examples, can partially obscure openings of one or more nanotubes 240 such that contact with markers primarily occurs on exterior surfaces of these nanotubes 240. However, contact along ends of these nanotube 240 with the electrodes can be irregular and can allow for a portion of the ends of these nanotubes 240 to be exposed, while a remainder of the nanotubes 240 could be in full contact and can be obscured. When markers bind or otherwise react with the surface of the nanotubes 240, the resistance of the nanotubes 240 changes. Typically, the resistance increases and the current decreases, although for some markers, resistance can decrease. FIGS. 2 and 3 show a readout for a positive and negative test result from the nanotube sensor of Working Example 3, which was substantially similar to the nanotube sensor 200, when exposed to a marker. Upon removal of the marker, resistance of the array 235 returns to initial levels.

In one exemplary embodiment, the nanotubes disclosed herein can be made of a metal oxide or a combination of several metal oxides. In one example, the metal oxide can be a transition metal oxide. In another optional example, the metal oxide can be a metal or semi-metal selected from Group 13 or 14 and can have an atomic number of 13 or greater (i.e. aluminum, silicon, gallium, germanium, indium, tin, thallium, and lead). Non-limiting examples of metal oxides that can be used to form the nanotubes include, but are in no way limited to, titanium dioxide, iron oxide, iridium oxide, tantalum oxide, zinc oxide, aluminum oxide, copper oxide, nickel oxide, chromium oxide, vanadium oxide, manganese oxide, zirconium oxide, palladium oxide, platinum oxide, cobalt oxide, lead oxide, silver oxide, tin oxide, magnesium oxide, and combinations thereof. In one example, the metal oxide can be titanium dioxide since titanium dioxide is self-ordering and binds or otherwise reacts with a large variety of markers. In another example, the metal oxide nanotubes can be formed of a single metal oxide. In one exemplary embodiment, the nanotubes disclosed herein can be formed from a non-metal oxide. For example, the nanotubes can include carbon nanotubes, even though carbon nanotubes are not self-ordering. However, the systems and methods for optimizing the use, functionality, and accuracy of the nanotube sensors can equally apply to non-metal oxide sensors. Another example of non-metal oxide materials that may form the nanotubes includes gold.

Typically, the metal oxide nanotubes are formed from anodized metal. For example, titanium dioxide nanotubes can be prepared, in some embodiments, by ultrasound assisted anodization. In one embodiment, a titanium foil anode and a platinum cathode can be used to form titanium dioxide nanotubes. Varying the anodization potential can control the diameter of the nanotubes, and changing the anodization time can vary the length of the nanotubes. Although dimension can vary for different materials and process conditions, the average diameter of the nanotubes can often range from about 20 nm to about 500 nm; the average lengths can often range from about 0.5 μm to about 50 μm; and the average wall thickness can range from about 1 nm to about 200 nm. The nanotubes can form ordered arrays of commonly aligned and oriented nanotubes. In one example, the array of nanotubes can be arranged with adjacent nanotubes substantially parallel to one another and stacked such that adjacent nanotubes are contacting one another. Ultrasonication during the anodization process can also result in improved ordering of the stacked nanotubes.

The ability or sensitivity of the nanotubes to detect the markers depends on or can be otherwise influenced by the diameter, length, and/or wall thickness of the nanotubes. In an exemplary embodiment, as previously discussed, the nanotubes disclosed herein can exhibit an average diameter of about 20 nm to about 500 nm. However, it is currently believed that nanotubes exhibiting an average diameter of about 50 nm to about 90 nm (e.g., about 50 nm to about 60 nm, about 55 nm to about 65 nm, about 60 nm to about 70 nm, about 65 nm to about 75 nm, about 70 nm to about 80 nm, about 75 nm to about 85 nm, or about 80 nm to about 90 nm) can be better able to detect certain markers and/or a wider variety of markers than nanotubes exhibiting other average diameters. As such, the average diameter of the nanotubes may be selected based on the marker or the variety of markers that the nanotubes are configured to detect. In an exemplary embodiment, as previously discussed, the nanotubes disclosed herein can exhibit an average length of about 0.5 μm to about 50 μm. However, it is currently believed that nanotubes exhibiting an average length of about 1.6 μm to about 2.1 μm (e.g., about 1.6 μm to about 1.8 μm, about 1.7 μm to about 1.9 μm, about 1.8 μm to about 2 μm, or about 1.9 μm to about 2.1 μm) can be better able to detect certain markers and/or a wider variety of markers than nanotubes exhibiting other average lengths. As such, the average length of the nanotubes may be selected based on the marker or the variety of markers that the nanotubes are configured to detect. In one exemplary embodiment, as previously discussed, the nanotubes disclosed herein can exhibit an average wall thickness of about 1 nm to about 200 nm. However, it is currently believed that nanotubes exhibiting an average wall thickness of about 4.5 nm to about 8.5 nm (e.g., about 4.5 nm to about 5.5 nm, about 5 nm to about 6 nm, about 5.5 nm to about 6.5 nm, about 6 nm to about 7 nm, about 6.5 nm to about 7.5 nm, about 7 nm to about 8 nm, or about 7.5 nm to about 8.5 nm) can be better able to detect certain markers and/or a wider variety of markers than nanotubes exhibiting other wall thicknesses. As such, the average wall thickness of the nanotubes may be selected based on the marker or the variety of markers that the nanotubes are configured to detect.

When the nanotubes include a metal oxide, the nanotubes can be annealed in oxygen to increase the electrical resistance of the nanotubes. For example, in one embodiment the as-anodized titanium dioxide nanotubes can be annealed in oxygen at 500° C. for 6 h to increase electrical resistance, although other temperatures and times can be used depending on the materials. As a general rule, annealing temperatures from about 200° C. to 600° C. can be used with annealing times from about 1 to 10 hours. Increasing the electrical resistance of the nanotubes can enhance the change of the resistivity of the nanotubes when the nanotubes bind or otherwise react with the marker.

Preferably, the nanotubes are non-functionalized such that a native surface of the nanotubes binds or otherwise reacts with a marker. For example, ammonia, nitrates, tuberculosis, tetrahydrocannabinol, and methamphetamine can be readily detected using non-functionalized metal oxide nanotubes, especially titanium dioxide nanotubes. Other markers can also be detected using non-functionalized nanotubes in a similar manner.

The non-functionalized nanotubes exhibit several benefits over functionalized nanotubes. In an example, the non-functionalize nanotubes are easier, quicker, and cheaper to manufacture since the nanotubes do not need to be functionalized. In an example, the non-functionalized nanotubes are more robust and exhibit longer shelf-lives. For instance, some functionalized nanotubes can bind or react with moisture in the air which makes the functionalized nanotubes less robust and exhibit shorter shelf-lives than non-functionalized nanotubes. In one example, the non-functionalized nanotubes can be more sensitive to a wider variety of markers than functionalized nanotubes. For instance, non-functionalized nanotubes can be able to accurately detect the presence of ammonia, nitrates, tuberculosis, tetrahydrocannabinol, and methamphetamine in air while functionalized nanotubes can be only able to detect one or none of ammonia, nitrates, tuberculosis, tetrahydrocannabinol, or methamphetamine. In one example, the non-functionalized nanotubes can be more reusable than functionalized nanotubes since it easier to remove the markers from the non-functionalized nanotubes than the functionalized nanotubes. For instance, functionalized nanotubes can bind or otherwise react more strongly with the markers than the non-functionalized nanotubes, thereby making removal of the markers from the functionalized nanotubes more difficult or impossible.

Regardless, in some embodiments, the nanotubes disclosed herein can be functionalized with at least one metal ion that is capable of binding with a specific marker. Non-limiting examples of metal ions that can be utilized to functionalize the disclosed nanotubes include Cu¹⁺, Li¹⁺, Fe²⁺, Ni²⁺, Cu²⁺, Co²⁺, Pb²⁺, Fe³⁺, Co³⁺, Cr³⁺, Mn³⁺, Ni³⁺, Sc³⁺, Sb³⁺, Ni⁴⁺, Mn⁴⁺, As⁴⁺, Sb⁴⁺, Pt⁴⁺, Zn²⁺, Pd²⁺, Ag¹⁺, and combinations thereof. In one alternative, the metal ions can be monovalent: Li¹⁺, divalent: Fe²⁺, Cu²⁺, Co²⁺, Pb²⁺, trivalent: Fe³⁺, Co³⁺, Cr³⁺, Mn³⁺, Ni³⁺, Sc³⁺, Sb³⁺, or tetravalent: Ni⁴⁺, Mn⁴⁺, Ti⁴⁺, As⁴⁺, Sb⁴⁺, Pt⁴. In one embodiment, the metal ion can include Co²⁺. In another example, the metal ion can include cobalt, chromium, copper, zinc, iron, nickel, palladium, gold, or combinations thereof. Although mixtures of ions can be used, in one example, the metal ions can be uniformly a single metal ion. Metal ions can be selected based on their ability to bind with a target marker. Computational modeling can be used to predict the affinities of various metal ions with various markers. Metal ions can also be tested experimentally using cyclic voltammetry methods as discussed in U.S. Pat. No. 10,241,078 issued on Mar. 26, 2019, the disclosure of which is incorporated herein, in its entirety, by this reference. Non-limiting examples of specific metal ion and marker pairs include chromium and methyl nicotinate, copper and glutathione, cobalt and glutathione, cobalt and biomarkers for tuberculosis (e.g., nicotinate and p-anisate), nickel and lactic acid, cobalt and lactic acid, nickel and tetrahydrocannabinol, cobalt and tetrahydrocannabinol, nickel and biomarkers for rectal cancer (e.g., cyclohexane, methylcyclohexane, 1,3-methylbenzene, and decanal), and the like.

The nanotubes can be functionalized with the metal ion or ions by metal ion exchange methods known in the art. Exchanging metal ions (Co, Zn, Cr, etc.) onto the titanium nanotube surface is made possible by the presence of large numbers of hydroxyl (Ti—OH) groups at the surface. These hydroxyl groups are exchangeable sites for binding metal ions. A surface hydroxyl proton is exchanged with a metal ion, binding the metal ion to the nanotube surface. Generally, the ion exchange can be performed by soaking the nanotubes in a solution containing the metal ion. In one embodiment, TiO₂ nanotubes can be functionalized with cobalt(II) ions by first heating the nanotubes to 100° C. to dehydrate the nanotubes, then soaking the nanotubes for 30 minutes in a solution of 0.5 wt % cobalt(II) chloride in ethanol, then rinsing the nanotubes and drying in a vacuum oven at 100° C. The time period for soaking the nanotubes in the metal ion solution can vary from about 30 minutes to about 5 hours.

In one example, the marker that the sensors detect can be determined in advance of the manufacturing of the nanotubes or sensor devices disclosed herein. For example, selection of the metal ions used in the functionalized nanotubes can be based on the marker(s) selected for detection. In one example, the marker that the sensors detect may not be determine in advance of manufacturing since, as will be discussed in more detail below, the sensors can be part of a system that includes machine learning algorithms and/or applies a large voltage differential to the nanotubes. As such, the sensors disclosed herein can be used to detect a variety of markers in a single test.

It is noted that any of the nanotube sensors disclosed herein may include other types of nanoparticles instead of or in addition to nanotubes. For example, any of the nanotube sensors disclosed herein may include nanodots, nano-bumps, nano-cones, nano-horns, or any other nanoparticles of varying geometries. However, it is noted that nanotubes may be more sensitive to the markers at least in part due to the increases surface area thereof compared to at least some of the other nanoparticles.

The nanotubes and the sensors disclosed herein can be utilized to detect a wide range of markers such as volatile organic compounds and/or non-volatile compounds. Accordingly, the sensor can be used to detect markers within a fluid, including both gaseous and liquid environments. Non-limiting examples of classes of markers that can be detected can include compounds associated with explosives, such as those associated with IED-type devices such as peroxides, nitrates, and the like, compounds associated with drinking water contamination such as trichloroethylene or arsenic, and compounds that are markers for a physiological condition or disease. Non-limiting examples of physiological conditions or diseases that can be diagnosed through the detection of associated volatile organic compounds in a subject's breath include, but are in no way limited to, tuberculosis (TB), breast cancer, lung cancer, heart disease, diabetes, preeclampsia, oxidative stress, COVID-19, Escherichia coli, listeria, and combinations thereof. When the volatile organic compound is a marker for a physiological condition or disease, the marker can be present in the breath of a subject. Thus, detection of the marker can be achieved by passing the expelled breath of the subject over the nanotubes in a sensor. Non-limiting examples of markers can include methyl phenylacetate, methyl p-anistate, methyl nicotinate, o-phenylanisole, lactic acid, reduced or oxidized glutathione, uric acid, urease, methamphetamine or derivatives thereof, tetrahydrocannobinol and derivatives thereof, and combinations thereof. Methyl phenylacetate, methyl p-anistate, methyl nicotinate, o-phenylanisole, are known markers for TB. Reduced and oxidized forms of glutathione are known markers for oxidative stress in a subject. Other markers that can be tested include, but are in no way limited to, trichloroethane, arsenic, selenium, and the like.

The sensors disclosed herein can have a power source that is configured to apply a voltage gradient to the nanotubes. As used herein, a voltage gradient is the absolute value of the maximum voltage applied to the nanotubes minus the minimum voltage applied to the nanotubes. The power source of the sensors disclosed herein can be configured to apply a relatively large voltage gradient to the nanotubes. The relatively large voltage gradient that is applied to the nanotubes allows the sensors disclosed herein to detect a variety of markers. For example, each marker can have a “sweet spot” where a change in voltage includes a change in the detected electrical current that is noticeably different compared to a substantially similar sample that does not include the marker. The relatively large voltage gradient applied to the array of nanotubes allows for a plurality of sweet spots for a plurality of different markers to be tested.

In an example, the relatively large voltage gradient that is applied to the nanotubes can be about 1 volt to about 12 volts, such as in ranges of about 1 volt to about 3 volts, about 2 volts to about 4 volts, about 3 volts to about 5 volts, about 4 volts to about 6 volts, about 5 volts to about 7 volts, about 6 volts to about 8 volts, about 7 volts to about 9 volts, about 8 volts to about 10 volts, about 9 volts to about 11 volts, or about 10 volts to about 12 volts. In such an example, the voltage applied to the nanotubes can exhibit a maximum voltage of about −2 volts to about 0 volts, about −1 volt to about 1 volt, about 0 volts to about 2 volts, about 1 volts to about 3 volts, about 2 volts to about 4 volts, about 3 volts to about 5 volts, or about 4 volts to about 6 volts and a minimum voltage of about 2 volts to about 0 volts, about 1 volt to about −1 volt, about 0 volts to about −2 volts, about −1 volts to about −3 volts, about −2 volts to about −4 volts, about −3 volts to about −5 volts, or about −4 volts to about −6 volts. Examples of voltages that can be applied to the nanotubes can be about −6 volts to 6 volts, about −4 volts to about 4 volts, about −3 volts to about 3 volts, about −2.5 volts to about 2.5 volts, about −2 volts to about 2 volts, about −1.5 volts to about 1.5 volts, about −1 volt to about 1 volt, about 0 volts to about 6 volts, about 0 volts to about 4 volts, or about 0 volts to about 3 volts.

The power source can be a direct current or alternating current power source. The power source includes at least one battery, at least one capacitor, at least one fuel cell, at least one solar cell, or other suitable power source. In some embodiments, power source is an internal component of the nanotube sensor such that the nanotube sensor can be a self-contained device. In other embodiments, the power source can be an external component of the nanotube sensor and can be connected to the housing through wires. In one exemplary embodiment, the power source is configured to apply a selected voltage to the nanotubes. However, it is noted that the power source can be configured to provide a selected electrical current to the nanotubes.

The power source may be configured to apply the voltage and/or current to the nanotubes over a time period that is about 1 second or greater, about 5 seconds or greater, about 10 seconds or greater, about 20 seconds or greater, about 30 seconds or greater, about 40 seconds or greater, about 50 seconds or greater, about 60 seconds or greater, about 70 seconds or greater, about 80 seconds or greater, about 90 seconds or greater, about 105 seconds or greater, about 120 seconds or greater, about 150 seconds or less, about 120 seconds or less, about 90 seconds or less, about 60 seconds or less, about 45 seconds or less, about 30 seconds or less, about 15 seconds or less, or in ranges of about 1 second to about 10 seconds, about 5 seconds to about 20 seconds, about 10 seconds to about 30 seconds, about 20 seconds to about 40 seconds, about 30 seconds to about 50 seconds, about 40 seconds to about 60 seconds, about 50 seconds to about 70 seconds, about 60 seconds to about 80 seconds, about 70 seconds to about 90 seconds, about 80 seconds to about 105 seconds, about 90 seconds to about 120 seconds, or about 105 seconds to about 150 seconds. The time period that the power source applies to the voltage and/or current to the nanotubes may depend on the range of voltages and/or current applied to the nanotubes. For example, applying a voltage to the nanotube in the range of about −3 volts to about 3 volts may take more time than applying a voltage to the nanotubes in a range of about −2 volts to about 2 volts.

As previously discussed, the nanotube sensor includes an electrical sensor (e.g., currently sensor). The electrical sensor can also be internal (e.g., disposed in or on the housing) or external (e.g., disposed outside of the housing). In one embodiment, the nanotube sensor can include the power source and the current sensor disposed outside of the housing. In such an embodiment, the nanotube sensor can be manufactured more cheaply and the portion of the nanotube sensor disposed in the housing can be disposed of or removed for sterilization after one or more uses, without disposing of the power source or current sensor. In some examples the power source and current sensor can be a single device that plugs into the housing of the nanotube sensor. Such a single device can include integrated controls or the single device can be configured to be controlled by a personal computer, laptop, smart phone, etc.

The electrical sensor may be connected to the nanotubes using a plurality of electrodes. The electrodes may be spaced apart from each other by a distance of about 0.1 cm to about 0.2 cm, about 0.15 cm to about 0.25 cm, about 0.2 cm to about 0.3 cm, about 0.25 cm to about 0.35 cm, about 0.3 cm to about 0.4 cm, about 0.35 cm to about 0.45 cm, about 0.4 cm to about 0.5 cm, about 0.45 cm to about 0.55 cm, about 0.5 cm to about 0.6 cm, about 0.55 cm to about 0.7 cm, about 0.6 cm to about 0.8 cm, about 0.7 cm to about 0.9 cm, about 0.8 cm to about 1 cm, about 0.9 cm to about 1.2 cm, about 1 cm to about 1.5 cm, about 1.25 cm to about 1.75 cm, about 1.5 cm to about 2 cm, about 1.75 cm to about 2.5 cm, about 3 cm to about 4, or greater than 4 cm. The distance between the electrodes affect the sensitivity of the nanotube sensor. For example, decreasing the distance between the electrodes will decrease the sensitivity of the electrical sensor detecting the changes in electrical resistance of the nanotubes. Increasing the distance between the electrodes may increase the noise detected by the electrical sensor. The distance between the electrodes may depend on the size of the surface area of the substrate that the nanotubes are formed on. In an embodiment, the electrodes may be printed on the nanotubes which may increase the consistency of the contact between the electrodes and the nanotubes than if the electrodes were formed on the nanotubes using clamps and soldered wires.

As previously discussed, the nanotubes sensors disclosed herein can include electrical circuitry. The electrical circuitry can be internal (e.g., disposed in or on the housing) or external (e.g., spaced from the housing). The electrical circuitry can include at least one processor and non-transitory memory. The non-transitory memory can include one or more operational instructions therein, and the at least one processor can be configured to execute the one or more operational instructions. In one exemplary embodiment, the one or more operational instructions include at least one machine-learning algorithm. The machine-learning algorithm can be configured to receive the electrical characteristics (e.g., electrical current) detected by the electrical sensor. The machine-learning algorithm can be configured analyze the electrical characteristics to determine if the electrical characteristics indicate the presence of one or more markers. As previously discussed, the markers that the machine-learning algorithm identifies can be previously known (e.g., the machine-learning algorithm is looking for the particular marker) or unknown (e.g., the machine-learning algorithm is looking for any possible markers). Responsive to the machine-learning algorithm's analysis, the electrical circuitry can output whether the sample included any markers. However, it is noted that the one or more operational instructions can include one or more non-machine-learning algorithms instead of, or in addition to, the machine-learning algorithm (e.g., an algorithm generated by a machine-learning algorithm).

It has been found that using machine-learning algorithms to determine whether a sample includes one or more markers optimizes the use, functionality, and accuracy of the nanotube sensors. For example, each marker can cause the electrical resistance to change when the marker bonds or otherwise reacts with a surface of the nanotubes. However, the change in the electrical resistance can be difficult to detect and it can be even more difficult to determine which marker caused the change in the electrical resistance. For example, the change in the electrical resistance can depend on how much (numerically) of the marker bonded or otherwise reacted with the surface and the marker can cause different changes in the electrical resistance at different voltages or electrical currents, both of which make detecting and identifying the marker more difficult. Additionally, other chemicals in the sample that are unimportant for a diagnostic perspective can also bond or otherwise react with the surface of the nanotubes. Such other chemicals can alter the electrical resistance of the nanotubes, thereby making the detection and identification of the markers more difficult (e.g., the other chemicals create noise or interference). Conventional nanotubes sensors attempted to overcome these difficulties by making the nanotubes highly specialized such that the conventional nanotubes can only detect one or only a few previously known markers. Such conventional nanotube sensors included highly specialized functionalized nanotubes that are configured to bond or otherwise react with only a specific marker and the electrical power (e.g., voltage) applied to the nanotubes has traditionally been applied at a very narrow range. However, it was discovered that using the machine-learning algorithm to analyze the detected electrical characteristics resolves the above indicated issues without requiring the nanotube sensors to exhibit a high specificity (e.g., only detect a single or only a few previously known markers), although the nanotubes sensors disclosed herein can be configured to exhibit a high specificity. For example, it was found that the machine-learning algorithms can detect a marker regardless of how much of the sample bonded or otherwise reacted with the nanotubes, whether the marker changes the electrical resistance at different voltages, or whether the sample included other chemicals with high accuracy. For example, the Working Examples provided below disclose that using about 800 training test data specimens is able to make the nanotube sensors disclosed herein 99.5% accurate, and increasing the number of training test data specimens was found to make the nanotube sensors disclosed herein approximately 100% accurate. Further, because of the machine-learning algorithms, the nanotube sensors disclosed herein can apply (e.g., voltage or current) exhibiting a wide range, even though the wide range of electrical power normally makes detecting the markers more difficult by increasing the noise detected. However, the wider range of electrical power and the fact that the machine-learning algorithm can detect the markers with additional noise allows the nanotube sensors disclosed herein to detect multiple markers and/or unknown markers.

In an embodiment, the machine-learning algorithms may be able to detect the presence of the one or more markers even when the electrical sensors detect significant amount of noise caused by variations between different nanotube sensors. For instance, variations in different nanotube sensors may cause the electrical characteristics detected by the electrical sensors of the different nanotube sensors to be different even when the same sample is supplied to the nanotube sensors. With sufficient number of samples, the machine-learning algorithm may be able to detect the noise caused by such variations even if the data used by the machine-learning algorithm was detected using one or more other nanotube sensors. Detecting the noise caused by such variations may allow the nanotube sensor to detect the presence of the markers in the samples supplied thereto even in view of the noise. In an example, the noise caused by variations between different nanotube sensors may be caused by variations in the quality and quantity of connection between the electrodes and the nanotubes. In an example, the noise caused by variations between different nanotube sensors may be caused variations in at least one of the diameter, length, or wall thickness of the nanotubes in the different nanotube sensors. Additionally, thermal considerations can induce noise in the system from sample to sample, and some creep of the baseline can occur. According to one example, the machine-learning algorithm can account for a change in the baseline signals (caused by noise, creep, connection variation, and other considerations) and can determine a delta from the determined baseline detecting air or an ambient/inert environment to perform the detection process. In some examples, the machine-learning algorithm can cause the system to perform a baseline check without a sample present to detect a variation in the baseline signals.

The non-transitory memory that includes the machine-learning algorithm can include a plurality of training test data specimens. The training test data specimens includes the electrical characteristics that were detected in previous tests, whether or not the tested sample included one or more markers, and the identity of the one or more markers if the tested sample included the marker. In an embodiment, the non-transitory memory can include about 100 or more training test data specimens, about 200 or more training test data specimens, about 400 or more training test data specimens, about 600 or more training test data specimens, about 800 or more training test data specimens, about 1000 or more training test data specimens, about 1250 or more training test data specimens, about 1500 or more training test data specimens, about 200 or more training test data specimens, about 2500 or more training test data specimens, about 3000 or more training test data specimens, about 4000 or more training test data specimens, about 5000 or more training test data specimens, about 7500 or more training test data specimens, about 10,000 or more training test data specimens, about 25,000 or more training test data specimens, about 50,000 or more training test data specimens, about 100,000 or more training test data specimens, about 250,000 or more training test data specimens, about 500,000 or more training test data specimens, about 1,000,000 or more training test data specimens or in range of about 100 training test data specimens to about 1000 training test data specimens, 500 training test data specimens to about 2000 training test data specimens, 1000 training test data specimens to about 10,000 training test data specimens, 5000 training test data specimens to about 50,000 training test data specimens, 10,000 training test data specimens to about 100,000 training test data specimens, 50,000 training test data specimens to about 500,000 training test data specimens, or 100,000 training test data specimens to about 1,000,000 training test data specimens. The number of training test data specimens that are stored on the memory can depend on a number of factors. In one example, the number of training test data specimens that are stored on the memory can depend on the desired accuracy of the nanotube sensor. For instance, as discussed in the Working Examples, about 850 training test data specimens are able to allow the nanotube sensors disclosed herein to detect methamphetamine in breath with about 99.5% accuracy. However, it has been found that increasing the number of training test data specimens to be greater than 2000 causes the nanotube sensors disclosed herein to detect methamphetamine in breath with approximately 100% accuracy. In an example, the number of training test data specimens stored in the memory can depend on the marker that is (or is not) present in training test data specimens since some markers can be more easily detected and identified compared to other markers, and thus, less training test data specimens are required for that particular marker. In one example, the number of training test data specimens used depends on the number of markers that the nanotube sensor is configured to detect, since increasing the number of markers that the nanotube sensor is configured to detect includes increasing the number of training test data specimens. In one example, the number of training test data specimens also depends on the size of the memory and the average size of the training test data specimens.

The machine-learning algorithm can be updatable. In one example, the nanotube sensor can be configured to receive additional training test data specimens, thereby allowing the machine-learning algorithm to be updated. In one example, the nanotube sensor can be configured to allow a user thereof to indicate whether the detection and identification of a marker was correct or wrong. For instance, a sample that is tested by the nanotube sensor can be tested by another nanotube sensor or another diagnostic test (e.g., gas chromatography mass spectrometer). The user of the nanotube sensor can then indicate whether the detection and identification of the marker was correct or wrong. The test data generated by the nanotube sensor can then become a training test data specimen.

The machine-learning algorithm can include any suitable machine-learning algorithm. For example, the machine-learning algorithm can include a supervised learning algorithm, an unsupervised learning algorithm, a reinforcement learning algorithm, a self-learning algorithm, a feature learning algorithm, a sparse dictionary learning algorithm, an anomaly detection algorithm, or an association rules algorithm. The machine-learning algorithm can include any suitable model, such as artificial neural networks (e.g., bi-directional long short-term neural networks), decision trees, support vector machines, regression analysis, Bayesian networks, training models, or federated learning models. In one example, the machine-learning algorithm can include ada boost from Python's scikit-learning package. In an example, the machining-learning algorithm can include a principle components of analysis (“PCA”) algorithm.

In an embodiment, the machine-learning algorithm can be selected based on the number of training test data specimens that are available. In an example, the machine-learning algorithm can be selected to be an ada boost algorithm when a relative low number of training test data specimens are available (e.g., less than 100) since the ada boost algorithm may be more accurate than at least some of the machine-learning algorithms in such an example. In an example, the machine-learning algorithm may be selected to be a PCA algorithm when a relatively medium number of training test data specimens are available (e.g., about 100 to about 1000) since the PCA algorithm may be more accurate than at least some of the machine-learning algorithms (e.g. ada boot algorithm) in such an example. In an example, the machine-learning algorithm may be selected to be an artificial neural network algorithm when a relative high number of training test data specimens are available (e.g., greater than about 1000) since the artificial neural network may be more accurate than at least some of the machine-learning algorithms (e.g., ada boot and PCA algorithms) in such an example.

In an embodiment, the machine-learning algorithm may include a plurality of different machine-learning algorithms. In such an embodiment, one, some, or all of the plurality of different machine-learning algorithms may be used to analyze a sample. The number and which of the plurality of machine-learning algorithms that are used to analyze the sample may be determined based on a number of factors. In an example, as previously discussed, the number and which of the plurality of machine-learning algorithms may be selected is based on the number of training test data specimens that are available since some machine-learning algorithms may be more accurate than others when a certain number of training test data specimens are available. In an example, using a plurality of machine-learning algorithms may be used to assess the accuracy of the analysis. For instance, the analysis by the plurality of machine-learning algorithms may be considered accurate when all the algorithms agreeing or substantially agreeing on the analysis. However, the analysis by the plurality of machine-learning algorithms may be considered less accurate when at least some of the algorithms have conflicting analyses.

In an embodiment, the nanotube sensor may be configured to detect the base line thereof. The baseline may refer to detecting the electrical characteristics of the nanotubes while exposed to either atmospheric air, an inert environment, or a uniform test sample (i.e., a sample having a known composition). The baseline may indicate noise that will be or may likely be detected by the electrical sensors when testing a sample with the nanotube sensor. The baseline may be unique for each nanotube sensor. The machine-learning algorithm may then use the baseline when analyzing any samples. For example, the machine-learning may subtract the baseline from what is detected by the electrical sensors when testing a sample thereby decreasing the noise that must be analyzed by the machine-learning algorithm and increasing the accuracy of the analysis.

It has been found that the baseline of the nanotube sensor may change over time. The baseline of the nanotube sensor may change due to markers becoming attached to the nanotube, fracturing or detaching the nanotubes from the substrate and/or electrodes due to the different thermal expansion coefficients thereof, or for other reasons. As such, the nanotube sensor may be configured to detect the baseline thereof after a certain number of samples (e.g., 5, 10, 15, or 20 samples) tested, after a certain period of time (e.g., after 1 hour), or after a certain process has been performed (e.g., after the nanotubes are heated or irradiated with UV light). Detecting the baseline after the certain number of samples, certain period of time, or after a certain process may improve the accuracy of the nanotube sensor. In an embodiment, the nanotube sensor may include one or more features to decrease the amount that the baseline of the nanotube sensors change. For example, the housing of the nanotube sensor may include a high thermal conductivity material and/or a heat sink to minimize the temperature of the nanotubes, the substrate, and the electrodes. Minimizing the temperature of the nanotubes, the substrate, and the electrodes may inhibit or prevent fracturing or detaching the nanotubes from the substrate and/or electrodes due to the different thermal expansion coefficients thereof.

In one exemplary embodiment, the one or more operation instructions of the non-transitory memory of the electrical circuitry includes at least one outlier detection algorithm. The outlier detection algorithm may analyze the training test data specimens to determine if the data contained in such specimens appears to be an anomaly (i.e., deviates from the other training test data specimens such that quality of the training test data specimen is suspicious). The outlier detection algorithm may omit any training test data specimen that appears to be an anomaly from the machine-learning algorithm's analysis. In an embodiment, the outlier detection algorithm may be a machine-learning algorithm such that the ability of the outlier detection algorithm to detect anomalies increases with the number of training test data specimens provided thereto. In an embodiment, the outlier detection algorithm may analyze electrical characteristics detected when the nanotubes were exposed to one or more samples to determine if such detected electrical characteristics should be included in the training test data specimens provided to the machine-learning algorithm.

The nanotube sensor can include a display connected to at least one of the electrical sensor or the electrical circuitry. The display is configured to display the result from the nanotube sensor in several ways. For example, the display can display a graphical representation of the current signal from the nanotube sensor. Alternatively, the display can simply indicate a “yes” or “no” to whether the target compound is present through an LED, an auditory buzzer, or the like.

In one exemplary embodiment, as previously discussed, the nanotube sensors disclosed herein can include a sample intake configured to direct flow of a sample (e.g., gas or fluid) over the nanotube array of the nanotube sensor. For example, the intake can be configured to sample ambient air or can be configured to receive a breath from a subject. In such embodiments, the air intake can include a particle filter configured to remove small particulate matter (PM10 and/or PM2.5) which can clog or otherwise limit the functionality and/or useful life of the nanotube sensor. The inlet can also include a concentrator configured to concentrate the air intake so as to increase the sensitivity of the nanotube sensor. A non-limiting example of a concentrator includes using solid extraction fibers which bind to volatile organic compounds that are then subsequently released. A molecular filter, charged chromatography column, and the like can also be used.

As previously discussed, the sensor can include a housing to contain the various components of the nanotube sensor. The filter, concentrator, and nanotube array can be oriented inside an interior space of the housing. The housing can contain the sample so that the sample can pass across and react with the nanotubes. The housing can also have an outlet for the sample to flow out from the housing. In one aspect, the intake and outlet can be disposed on opposite sides of the nanotube array so that the sample flows across the nanotube array. In embodiments where the sample is expired breath from a subject, the subject can breathe into the intake. The intake can include a mouthpiece configured in size and shape to comfortably fit into the mouth of the subject to allow the subject to breath into the sensor. In some embodiments the intake can also include a one-way valve to prevent backflow of gases out through the intake. The mouthpiece can optionally be disposable or replaceable and can be configured to engage with the intake. The outlet can also include a valve that allows air to pass through when the subject is blowing, but then prevents air from escaping from the housing during the testing period. In this way, the expired breath, and the markers therein, can be prevented from flowing or diffusing out of the housing during testing. In other embodiments, the sample can be recirculated across the nanotubes, such that the markers will have additional opportunity to bind or otherwise react with the nanotubes.

In one exemplary embodiment, the nanotube sensors disclosed herein are configured to be reusable. In such an embodiment, the nanotube sensors can be configured to allow the nanotubes to be exposed to a stimulus that causes at least some of the markers to be released from the nanotubes. In one example, the stimulus that releases the markers from the nanotubes is ultraviolet light. In such an example, the nanotube sensor can include an ultraviolet light source disposed in the housing or the housing can be at least partially transparent to ultraviolet light such that an external ultraviolet light source can illuminate the nanotubes. In one example, the stimulus that releases the markers from the nanotubes includes heating the nanotubes to a selected temperature (e.g., a temperature of about 300° F. or greater, about 400° F. or greater, or about 500° F. or greater) for a time period sufficient to release the marker (e.g., about 1 hour or more, about 2 hours or more, about 3 hours or more, about 4 hours or more, about 5 hours or more, or about 6 hours or more). In such an example, the housing and the components disposed in the housing are configured to be exposed to a temperature that is sufficient to release the marker from the nanotube array. It is noted that the nanotube sensor can be exposed to the stimulus after each use or may only be exposed to the stimulus after multiple uses of the nanotube sensor (e.g., when the nanotubes are saturated or nearly saturated with markers). In one exemplary embodiment, the nanotube sensors disclosed herein are configured to be disposable. When configured to be disposable, the nanotube sensors can be made of inexpensive materials including some or all of the materials being biodegradable.

In one exemplary embodiment, a method of detecting the markers is provided. The method includes the steps of exposing the nanotube array to a sample (e.g., gaseous sample) such that one or more markers can bind with the nanotubes. During or after exposing the nanotube array to the sample, the method includes applying a plurality of voltages across a nanotube array, measuring an electrical current through the nanotube array at each voltage, and analyzing the detected electrical current, such as with the machine learning algorithms, to determine if the one or more markers were detected by the nanotube sensor. Determining if the one or more markers were detected can include identifying the marker if the marker is not already known.

In one exemplary embodiment, the method of detecting the one or more markers includes determining a baseline. Determining the baseline can include, before exposing the nanotube array to the sample, applying the plurality of voltages across a nanotube array and measuring the electrical current through the nanotube array at each voltage. Determining if the one or more markers were detected by the nanotube sensor can include comparing the electric current detected during the baseline with the electric current detected during or after exposing the nanotube array to the sample.

In one embodiment, the method can further include the step of diagnosing the human subject with a physiological condition or disease based on the identifying of the marker. In one exemplary embodiment, the method can further include exposing the nanotubes to a stimulus when the nanotube sensor is configured to be reusable.

The following working examples provide further detail about the nanotube sensors disclosed herein, in accordance with the principles of some of the specific embodiments disclosed herein.

### Working Example 1

Working Example 1 includes a nanotube sensor that included a thin layer of a plurality of titanium dioxide nanotubes on a small titanium metal coupon. The plurality of titanium dioxide nanotubes exhibited a diameter of about 50 nm and length of about 100 nm to about 200 nm. The nanotube sensor included an external potentiostat plugged into a laptop.

The nanotube sensor of Working Example 1 was tested to determine if atmospheric methamphetamine could be detected by the nanotube sensor. The nanotube sensor of Working Example 1 was tested by placing the nanotube sensor in the flow path of gases discharged from a gas chromatography mass spectrometer routinely used by the Utah Forensic Services labs specifically for the detection of methamphetamine.

A plurality of samples were provided. Some of the samples included methamphetamine and a remainder of the samples did not include methamphetamine A small quantity of the each sample was pipetted and sprayed into the gas chromatography mass spectrometer one at a time. When the sample entered the gas chromatography mass spectrometer, the sample was evaporated, pumped through the analysis instrumentation of the gas chromatography mass spectrometer to determine if the sample included methamphetamine, and then discharged from the gas chromatography mass spectrometer. The sample that was discharged from the gas chromatography mass spectrometer was then received by the nanotube sensor of Working Example 1. The plug-in potentiostat controlled the voltage applied to the nanotube array of the nanotube sensor and measured the corresponding electrical current.

The electrical current measured by the potentiostat was compared to the readings of the gas chromatography mass spectrometer to determine if the nanotube sensor of Working Example 1 detected the methamphetamine Comparing the results showed that the electrical current detected by the potentiostat was noticeably different depending on whether the sample included methamphetamine or not. Thus, the test of the nanotube sensor of Working Example 1 demonstrated that the nanotube sensors disclosed herein can detect atmospheric methamphetamine.

### Working Example 2

Working Example 2 included two nanotube sensors that were substantially the same as the nanotube sensor of Working Example 1. The two nanotube sensors of Working Example 2 were tested to simulate a field test.

A plurality of markers were provided. Marker 1 included methamphetamineanol only, marker 2 included breath only, marker 3 included air only, marker 4 included a solution of 1 mg of methamphetamineyl p-anisate per 1 mL of methamphetamineanol, marker 5 included a solution of 0.1 mg of methamphetamineyl nicotinate per 1 mL of methamphetamineanol, and marker 6 included a solution of 1 mg of methamphetamineamphetamine per 1 mL of methamphetamineanol. 20 gaseous samples of each of the markers were formed in sample bags. The gaseous samples that includes markers, 1, 4, 5, and 6 were formed by injecting 2 mL of each marker into the sample bag and then air or breath was added to the sample bag.

Each of the two nanotube sensors were exposed to 10 of the gaseous samples for each markers. Each of the two sensors corrected identified the markers with 100% accuracy.

### Working Example 3

Prior research and publications have indicated that there is a voltage ‘sweet spot’ for each marker, such as tuberculosis, marijuana's tetrahydrocannabinol, and cocaine's methamphetamine Finding the sweet spot for each marker can be difficult, and this problem is further complicated by the fact that each marker can include more than one, albeit less sensitive, sweet spot.

To find the sweet spot for methamphetamine, the nanotube sensor of Working Example 1 was exposed to a first sample that included only air followed by a second sample that included methamphetamine in air. A plurality of voltages were applied to the nanotube array of the nanotube sensor across all of the possible sweet spots while the electrical current was detected. FIG. 2 is a graph illustrating the detected current of a sample of Working Example 3 that included methamphetamine FIG. 3 is a graph illustrating the detected current of a sample of Working Example 3 that included methamphetamine and a sample of Working Example 3 that did not include methamphetamine. As shown in FIG. 3, the detected current of the samples that included methamphetamine was noticeably different than the detected current of the samples that did not include methamphetamine.

The nanotube sensor was further tested by exposing the nanotube sensor to the first or second samples while one or more background volatiles were added to try to confound the patterns and detection accuracies. The background volatiles included variations of air only, air plus methamphetamineanol, isopropyl alcohol, breath, methamphetamineanol, methamphetamineanol plus p-anisate and methamphetamineanol plus nicotinate. A total of 1071 tests were performed.

The machine-learning algorithm ada-boost from python's scikit-learn package was used to analyzed the results and successfully classified those otherwise hidden patterns that indicate methamphetamine in the detected experimental current-voltage traces. For example, the machine-learning algorithms were trained by randomly providing 856 of the test results to the machine-learning algorithm. The remaining 215 test results were then provided to determine whether the machine-learning algorithm could detect whether the sample that generated the test results included methamphetamine or not. The machine-learning algorithm was able to correctly determine whether the sample that generated the test results included methamphetamine or not with 99.5% accuracy.

### Prophetic Example 1

Molecular modeling and quantum chemical calculations evaluated the theoretical influence of methamphetamine on the surface of the nanotubes. The computer packages Avogadro, Mopac, Gabedit, Vesta, and Quantum Espresso were used to analyze three different categories of molecular modeling and quantum chemical calculations. The first category of molecular modeling and quantum chemical calculations was force field calculations to find energy-minimized molecular configurations. The second category of molecular modeling and quantum chemical calculations was ab initio quantum calculations made on finite, discrete numbers of atoms and molecules. The third category of molecular modeling and quantum chemical calculations was Density Functional Theory (DFT) calculations made on infinite, continuous slabs of sensor atoms.

The results of the molecular modeling and quantum chemical calculations of Prophetic Example 1 were positive. The molecular modeling and quantum chemical calculations correctly predicted that the samples that includes methamphetamine and the samples that did not include methamphetamine would cause different electrical currents to be detected, as demonstrated in Working Examples 1 to 3.

### Prophetic Example 2

Prophetic Example 2 first included calculating an energy-minored molecular configured and structure of methamphetamine. Then, Prophetic example 2 included calculating the frontier orbitals and associated chemical descriptors of the methamphetamine and the titanium dioxide nanotubes. The frontier orbitals calculated included the highest occupied molecular orbital (HOMO), lowest unoccupied molecular orbital (LUMO), the absolute value of the HOMO of the methamphetamine minus the LOMO of titanium dioxide nanotubes (H-L), and the absolute value of the LOMO of the methamphetamine minus the HOMO of titanium dioxide nanotubes (L-H). The associated chemical descriptions included electron affinity (A), ionization potential (B), electro-negativity (C), chemical hardness (D), degree of electron transfer (E), and degree of energy transfer (F). How each of the chemical descriptions were calculated are shown in Table 1.

TABLE 1

| | |
|---|---|
| electron affinity | A = −LUMO |
| ionization potential | B = −HOMO |
| electro-negativity | C = (A + B)/2 |
| chemical hardness | D = (A − B)/2 |
| electron transfer | E = 0.5*abs[C(1) − C(2)]/[D(1) − D(2)] |
| energy transfer | F = 0.25*abs[(C(1) − C(2)]\hat{ }2/[D(1) + D(2)] |

The calculated frontier orbitals and associated chemical descriptors are shown in Table 2.

TABLE 2

| ev | H (homo) | L (lumo) | H-L | L-H | A | B | C(1, 2) | C1-C2 | D(1, 2) | D1-D2 | E | D1 + D2 | F |
|---|---|---|---|---|---|---|---|---|---|---|---|---|---|
| meth | −8.56 | 0.32 | | | −0.32 | 8.56 | 4.12 | | −4.44 | | | | |
| TiO2 | −12.00 | −7.86 | 0.70 | 12.32 | 7.86 | 12.00 | 9.93 | −5.81 | −2.07 | −2.37 | 1.23 | −6.51 | 6.88 |

Table 2 shows a relatively large difference in C(1) and C(2), the electro-negativity for the methamphetamine and the titanium dioxide nanotubes, respectively, which suggests a strong interaction between the methamphetamine and the titanium dioxide nanotubes. By contrast, it was found that the methamphetamine and the titanium dioxide nanotubes exhibit a relatively small difference in chemical hardness, D(1) and D(2). The relatively small difference in chemical hardness indicates a stronger interaction between the methamphetamine and the titanium dioxide nanotubes because the ‘Hard-Soft Acid-Base’ theory states that ‘hard likes hard and soft likes soft’. Both the methamphetamine and the titanium dioxide nanotubes are relatively ‘hard’ and, thus, liked each other.

Furthermore, Table 2 shows a small energy gap (0.70 eV) between the titanium dioxide nanotubes HOMO and the methamphetamine's LUMO. This suggested a higher reactivity because it was easier for the titanium dioxide nanotube's HOMO electrons to occupy or covalently ‘share’ the methamphetamine's vacant LUMO orbitals. In addition, the relatively large electron transfer (E) and energy transfer (F) indicated a strong interaction processes and good sensor reliability. Other surface species such as phenols or carboxylates act as contaminants and also come into play. These contaminants tend to lower or change the energy gap between the methamphetamine and the titanium dioxide nanotubes, and can thus confound and confuse classification of the sensor reading. Large and diverse training sets for the classifier were therefore necessary.

Prophetic Example 2 also included calculating the mutual methamphetamine-titanium dioxide nanotube attraction from the overall charge differences between the methamphetamine and the surface of the titanium dioxide nanotubes. First a complex spatial variation of the methamphetamine molecule was found by first calculating the molecular wave function. The complex spatial variations of the methamphetamine resulted in a description of nanotube-marker interaction which suggested good sensor quality and reliability.

Prophetic Example 2 also included calculating the density of states (DOS). The DOS of methamphetamine is empty and has a large number of empty states above its LUMO of 0.32 ev. By contrast the DOS of the titanium dioxide nanotubes are full and has a large number of full states below its HOMO of −8.56 ev. Electrons in the titanium dioxide nanotubes overcrowded ‘apartments’ were ready to fill or covalently share the empty apartments or spaces provided by the methamphetamine. The titanium dioxide nanotubes acted as a Lewis base that wanted to hook up with and give electrons to the Lewis acid, methamphetamine.

Prophetic Example 2 also included calculating the fermi levels which are a powerful indicator of titanium dioxide nanotubes quality and reliability of detecting methamphetamine. The fermi level calculations came from Density Functional Theory (DFT) calculations based on an infinite slab of titanium dioxide. Fermi levels were calculated in the absence or presence of the methamphetamine and showed a change of 0.07 ev due to the presence of methamphetamine. This meant a theoretical change of 0.07 volt in the small titanium sensor, and this was good enough to produce over 99% classification accuracy.

Working Examples 1 to 3 and Prophetic Examples 1 and 2 showed that the nanotube sensors disclosed herein detects methamphetamine in off gases without heat or other sample manipulation. These results also correlated with theoretical frontier orbital and fermi level calculations.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, ±5%, +2% or 0% of the term indicating quantity. In an example, when the term of degree is used to modify a shape, the term of degree indicates that the shape being modified by the term of degree has the appearance of the disclosed shape. For instance, the term of degree may be used to indicate that the shape can have rounded corners instead of sharp corners, curved edges instead of straight edges, one or more protrusions extending therefrom, is oblong, is the same as the disclosed shape, etc.

We claim:

1. A nanotube sensor, comprising:
an array of nanotubes formed on a substrate;
a power source connected to and configured to apply electrical power to the array of nanotubes;
an electrical sensor configured to detect at least one electrical characteristic when the electrical power is provided to the array of nanotubes, the at least one electrical characteristic including at least one of a voltage of, an electrical current flowing through, or electrical resistance of the array of nanotubes; and
electrical circuitry communicably coupled to the electrical sensor configured to receive the at least one electrical characteristic from the electrical sensor, the electrical circuitry including non-transitory memory including at least one machine-learning algorithm and at least one processor, wherein the at least one processor is configured to input the at least one electrical characteristic into the at least one machine-learning algorithm and execute the at least one machine learning algorithm to determine if the array of nanotubes was exposed to a marker.

2. The nanotube sensor of claim 1, wherein the array of nanotubes comprises an array of non-functionalized nanotubes.

3. The nanotube sensor of claim 1, wherein the array of nanotubes comprises an array of functionalized nanotubes.

4. The nanotube sensor of claim 1, wherein the array of nanotubes comprises titanium dioxide.

5. The nanotube sensor of claim 1, wherein the array of nanotubes exhibits at least one of an average diameter of about 50 nm to about 90 cm, an average length of about 0.5 μm to about 50 μm, or an average wall thickness of about 4.5 nm to about 8.5 nm.

6. The nanotube sensor of claim 1, wherein the non-transitory memory includes at least 800 training test data specimens.

7. The nanotube sensor of claim 1, wherein the at least one machine-learning algorithm comprises a principle components of analysis algorithm.

8. The nanotube sensor of claim 1, wherein the at least one machine-learning algorithm comprises an artificial neural network.

9. The nanotube sensor of claim 1, wherein the at least one machine-learning algorithm comprises a plurality of machine-learning algorithms.

10. The nanotube sensor of claim 1, wherein the non-transitory memory comprises an outlier detection algorithm.

11. The nanotube sensor of claim 1, further comprising at least one of an ultraviolet light source or a heater configured to release the marker from the array of nanotubes.

12. A method of using a nanotube sensor, the method comprising:
flowing a sample over an array of nanotubes formed on a substrate;
applying electrical power to the array of nanotubes with a power source that is connected to the array of nanotubes;
while the power source is applying electrical power to the array of nanotubes, detecting at least one electrical characteristic with an electrical sensor, the at least one electrical characteristics including at least one of a voltage of, an electrical current flowing through, or electrical resistance of the array of nanotubes; and
with at least one processor of an electrical circuitry, inputting the at least one electrical characteristic into at least one machine-learning algorithm and executing the at least one machine learning algorithm to determine if the array of nanotubes was exposed to a marker, the at least one machine-learning algorithm included in non-transitory memory of the electrical circuitry, the electric circuitry communicably coupled to the electrical sensor and configured to receive the at least one electrical characteristic from the electrical sensor.

13. The method of claim 12, wherein applying electrical power to the array of nanotubes with a power source that is connected to the array of nanotubes includes applying a voltage of about −3 volts to about 3 volts to the array of nanotubes.

14. The method of claim 12, wherein executing the at least one machine learning algorithm to determine if the array of nanotubes was exposed to a marker includes executing the at least one machine learning algorithm to determine if the array of nanotubes was exposed to a plurality of markers.

15. The method of claim 12, wherein executing the at least one machine learning algorithm to determine if the array of nanotubes was exposed to a marker executing the at least one machine learning algorithm to determine if the array of nanotubes was exposed to a marker indicative of severe acute respiratory syndrome coronavirus 2.

16. The method of claim 12, wherein executing the at least one machine learning algorithm to determine if the array of nanotubes was exposed to comprises marker comprises executing the at least one machine learning algorithm to determine if the array of nanotubes was exposed to a marker indicative of at least one of tuberculosis, Escherichia coli, listeria, methamphetamine, explosive compounds, colorectal cancer, ammonia, nitrates, or tetrahydrocannabinol.

17. The method of claim 12, wherein executing the at least one machine learning algorithm comprises executing at least one of a principle components of analysis algorithm or an artificial neural network.

18. The method of claim 12, further comprising, obtaining a baseline by:
flowing atmospheric air or a uniform testing sample over the array of nanotubes;
applying electrical power to the array of nanotubes with the power source; and
while the power source is applying electrical power to the array of nanotubes, detecting the at least one electrical characteristic of the array of nanotubes with the electrical sensor.

19. The method of claim 18, further comprising obtaining the baseline after flowing the sample over the array of nanotubes.

20. A nanoparticle sensor, comprising:
an array of nanoparticles formed on a substrate;
a power source connected to and configured to apply a voltage to the array of nanoparticles;
an electrical sensor configured to detect at least one electrical characteristic when the voltage is provided to the array of nanoparticles, the at least one electrical characteristic including at least one of an electrical current flowing through or electrical resistance of the array of nanoparticles; and
electrical circuitry communicably coupled to the electrical sensor and configured to receive the at least one electrical characteristic from the electrical sensor, the electrical circuitry including non-transitory memory including at least one machine-learning algorithm and at least one processor, wherein the at least one processor is configured to input the at least one electrical characteristic into the at least one machine-learning algorithm and execute the at least one machine learning algorithm to determine if the array of nanoparticles was exposed to a marker.
